(19) United States
(12) Patent Application Publication
(10) Pub. No.: US 2020/0407310 A1
(43) Pub. Date: Dec. 31, 2020

(54) PROCESS FOR PURIFYING HEXAMETHYLENEDIAMINE

(71) Applicant: BASF SE, Ludwigshafen (DE)

(72) Inventor: Fabien RIVOLLET, Caluire et Cuire (FR)

(73) Assignee: BASF SE, Ludwigshafen (DE)

(21) Appl. No.: 16/975,817

(22) PCT Filed: Feb. 27, 2019
(86) PCT No.: PCT/EP2019/054845

§ 371 Date: Aug. 26, 2020

(30) Foreign Application Priority Data
Feb. 27, 2018 (EP) 18305205.9

(51) Int. Cl. C07C 209/84; C07C 209/48; B01D 3/14

(52) U.S. Cl. CPC C07C 209/84; C07C 211/12; B01D 3/141; C07C 209/48

(57) ABSTRACT

A process for purifying and/or preparing hexamethylenediamine by hydrogenating adiponitrile in the presence of a hydrogenation catalyst and purifying the thus obtained mixture of hexamethylenediamine, impurities, and water.

### TECHNICAL FIELD OF THE INVENTION

The present invention relates to a process for purifying hexamethylenediamine. The invention further relates to a process for the preparation of hexamethylenediamine by hydrogenating adiponitrile in the presence of a hydrogenation catalyst and purifying the thus obtained mixture comprising hexamethylenediamine, impurities and water.

### BACKGROUND

Hexamethylenediamine (HMD) is a chemical intermediate of great importance, used in particular as monomer in the manufacture of polyamides.

For example, hexamethylenediamine is used in combination with adipic acid to form an amine salt, hexamethylenediamineadipate, also known as Nylon salt. This salt is employed in the manufacture of a poly(hexamethyleneadipamide), more commonly known as PA 6,6.

Hexamethylenediamine is also an important chemical intermediate, for example in the manufacture of diisocyanate compounds.

The process for the manufacture of hexamethylenediamine used industrially, involves hydrogenating adiponitrile in the presence of a hydrogenation catalyst, such as a metal catalyst of Raney type, such as Raney nickel or Raney cobalt.

After the hydrogenation process, the hydrogenated products and, in particular hexamethylenediamine, are usually recovered by a sequence of distillations intended to remove water and impurities. Such impurities are generated by the decomposition of certain compounds or by reaction between the molecules present.

Major known impurities are hexamethyleneimine (HMI), diaminocyclohexane (DCH), aminocyclopentanemethylamine, bishexamethylentriamine (BHT) and other imines, such as tetrahydroazepine (THA) and oligomers composed of the imines and HMD, as well as water.

The majority of these impurities are a hindrance in the use of hexamethylenediamine, for example as monomer in the manufacture of polyamides. This is because they can generate impurities in the polyamide obtained, causing a yellow coloring of the latter and inhomogeneities in the material, which bring about defects and breakages, in particular during the manufacture of yarns.

Numerous processes have been proposed for purifying hexamethylenediamine, thereby obtaining the desired diamine with a high degree of purity. However, these processes require a number of subsequent distillation steps wherein high boiling and low boiling impurities are removed one after another. The known processes are therefore time and energy consuming and require much equipment, such as several distillation columns, which makes the processes expensive and environmentally unfriendly.

In particular, in the production of polyamides, such as PA 6,6, for the manufacture of yarns and textile fibers, it is desirable to have hexamethylenediamine of high purity. There is therefore still a need for further improvements in the purification of hexamethylenediamine.

### SUMMARY

The present inventors now found that many of the known impurities which are obtained in the manufacture of hexamethylenediamine can surprisingly be removed in a single distillation step, if the hexamethylenediamine mixture comprising hexamethylenediamine and the impurities is subjected to distillation in a dividing wall column (Petlyuk column).

One aspect of the present invention therefore provides a process for purifying hexamethylenediamine, wherein a mixture comprising hexamethylenediamine and impurities is subjected to distillation in a dividing wall column (Petlyuk column) and the purified hexamethylenediamine is recovered from a side stream of the dividing wall column (Petlyuk column), and wherein the mixture prior to distillation comprises impurities at an amount such that the mixture has a UV index (iUV) of at least 0.10 and the purified hexamethylenediamine has an iUV of 0.08 or less.

Furthermore, the present inventors found that, under certain conditions, alkanolamines, such as 1,2-aminocyclohexanol (ACHOL) are formed as impurities. The inventors furthermore found that this additional, previously not described by-product of the nitrile hydrogenation also can cause problems in the further use of the desired diamine. Like, for example, diaminocyclohexane also 1,2-aminocyclohexanol can cause in-homogeneities in polyamides manufactured from the diamine. It is therefore desirable to also remove the 1,2-aminocyclohexanol from a mixture comprising hexamethylenediamine and the 1,2-aminocyclohexanol.

The present inventors found that also 1,2-aminocyclohexanol can be effectively separated from a mixture comprising hexamethylenediamine and impurities by distillation in a dividing wall column (Petlyuk column).

Another aspect of the present invention therefore provides a process for purifying hexamethylenediamine wherein the mixture comprises from about 10 to about 5,000 ppm of 1,2-aminocyclohexanol.

Another aspect of the present invention relates to a process for the preparation of hexamethylenediamine by hydrogenating adiponitrile in the presence of a hydrogenation catalyst to obtain a mixture comprising hexamethylenediamine, impurities and water, and purifying the obtained mixture by the above process.

### DETAILED DESCRIPTION

The invention relates to a process for purifying hexamethylenediamine, wherein a mixture comprising hexamethylenediamine and impurities is subjected to distillation in a dividing wall column (Petlyuk column) and the purified hexamethylenediamine is recovered from a side stream of the dividing wall column (Petlyuk column), and wherein the mixture prior to distillation comprises impurities at an amount such that the mixture has a UV index (iUV) of at least 0.10 and the purified hexamethylenediamine has an iUV of 0.08 or less.

A characteristic feature of the purity of hexamethylenediamine is expressed in the form of its UV index (iUV). This index is obtained by measuring the UV absorbance at a wavelength of 275 nm of a 32.4% by weight solution of the mixture comprising hexamethylenediamine and impurities in water in a cell with a length of 5 cm. Mixtures comprising hexamethylenediamine and impurities as obtained for example from the hydrogenation of adiponitrile can, for example, have an iUV of at least 0.10, such as at least 0.20 or even at least 0.50. The present inventors found that the impurities and thus this index can be reduced by a single distillation in a dividing wall column (Petlyuk column) to 0.08 or less, preferably 0.07 or less, more preferably 0.06 or less and even more preferably 0.05 or less.

Typical impurities in the mixture fed to the dividing wall column (Petlyuk column) in the process of the invention comprise hexamethyleneimine, diaminocyclohexane, aminocyclopentanemethylamine, bishexamethylenetriamine, 1,2-aminocyclohexanol, and other imines, such as tetrahydroazepine, and oligomers of imines and hexamethylenediamine, and any mixtures thereof.

A particular impurity is 1,2-aminocyclohexanol, which can be present in the mixture in an amount of up to about 5,000 ppm, such as about 10 to about 5,000 ppm, preferably from about 10 to about 400 ppm, based on the weight of the hexamethylenediamine in the mixture.

If the term “about” is used herein before a quantitative value, the present teachings also include the specific quantitative value itself, unless specifically stated otherwise. As used herein, the term “about” refers to a ±10% variation from the nominal value unless specifically stated otherwise.

The process of the invention is also effective in removing 1,2-aminocyclohexanol from the mixture comprising hexamethylenediamine and impurities. For example, the hexamethylenediamine purified by the process of the invention can comprise less than 8 ppm, preferably less than 5 ppm and even more preferably less than 2 ppm of 1,2-aminocyclohexanol.

The inventors furthermore found that a low amount of water in the mixture prior to distillation facilitates the purification of hexamethylenediamine. In a preferred embodiment, the mixture comprises less than 5,000 ppm of water, preferably less than 3,000 ppm of water, more preferably less than 1,000 ppm of water, even more preferably less than 500 ppm of water.

The invention is based on the finding that a mixture comprising hexamethylenediamine and impurities can be purified in a single step by distillation in a dividing wall column (Petlyuk column). In such one-step purification process, the impurities and, in particular, those impurities characterized by the UV index of the mixture, are simultaneously removed at the top and the bottom of the column. Thus, the high boiling impurities (those impurities having a higher boiling point than hexamethylenediamine) are removed at the bottom of the column, the low boiling impurities (those impurities which have a lower boiling point than hexamethylenediamine) are removed from the top of the column, and the purified hexamethylenediamine is recovered from a side stream of the column.

The process of the invention thus has the advantage over the prior art processes that only one instead of several distillation columns are required. This not only saves time and equipment but additionally can save up to about 20% of the steam consumption in comparison to known processes using at least two columns for subsequent removal of the low boiling impurities and the high boiling impurities.

In one embodiment, the top pressure of the dividing wall column (Petlyuk column) used in the process of the invention is between about 0.1 and about 25 kPa.

In another embodiment, the bottom pressure of the dividing wall column (Petlyuk column) used in the process of the invention is between about 0.1 and about 40 kPa. The bottom pressure is selected such that it is higher than the top pressure.

In a further embodiment, the total number of theoretical plates of the dividing wall column (Petlyuk column) used in the process of the invention is at least about 50, preferably between about 50 and about 200.

The distillation parameters like distillate and bottom rates, can preferably be adjusted such that at least about 75%, preferably at least about 80% of the hexamethylenediamine fed into the dividing wall column (Petlyuk column) is recovered as purified hexamethylenediamine, in particular from a side stream of the column.

Preferably the feed flow of the dividing wall column (Petlyuk column) is in the form of a vapor phase.

The process of the present invention is particularly suitable for separating impurities from a mixture comprising hexamethylenediamine, impurities and water, wherein said mixture is obtained during the manufacture of hexamethylenediamine from adiponitrile by hydrogenation. The present invention therefore also relates to a process for the preparation of hexamethylenediamine which comprises the steps of
a) hydrogenating adiponitrile in the presence of a hydrogenation catalyst to obtain a mixture comprising hexamethylenediamine, impurities and water,
b) optionally removing at least part of the water from the mixture obtained in step a),
c) optionally removing part of the impurities having a higher boiling point than hexamethylenediamine from the mixture obtained in step a) orb); and
d) purifying the mixture obtained in step a), b) or c) by the above described process.

The mixture comprising hexamethylenediamine, impurities and water obtained by hydrogenating the adiponitrile can either be separated directly by the above described process or, alternatively, can be subjected to further process steps, such as purification steps, prior to the purification process of the present invention.

For example, it can be advantageous to remove at least part of the water from the crude mixture obtained from the hydrogenation step so that the mixture comprises less than 5,000 ppm of water before the purification process of the invention is carried out. The water can be removed using methods known in the art, such as distillation.

It can furthermore be advantageous to remove part of the impurities having a higher boiling point than hexamethylenediamine from the mixture prior to the purification process of the invention. Removing part of the impurities having a higher boiling point than hexamethylenediamine can be conducted by usual methods known in the art, for example by distillation.

Preferably, the process for the preparation of hexamethylenediamine according to the invention comprises steps a), b), c) and d).

It is also advantageous, in order to limit the losses of hexamethylenediamine, to treat the fractions comprising the high boiling impurities obtained from the dividing wall column (Petlyuk column) and/or from the previous removal of part of the high boiling impurities. This treatment can be carried out in a conventional distillation column with distillation of the hexamethylenediamine or in columns of thin film evaporation type.

Alternatively, at least part of the fraction from the bottom of the dividing wall column (Petlyuk column) in above step d) and/or at least part of the fraction comprising impurities having a higher boiling point than hexamethylenediamine obtained in above step c) are treated to recover hexamethylenediamine, and the recovered hexamethylenediamine is recycled in any step after step a).

If in the above process step c) is conducted, it is furthermore advantageous to feed the mixture obtained in step c) into the dividing wall column (Petlyuk column) of step d) in the form of its vapor phase. In this case, the overall energy consumption of the process can be reduced.

The partial or complete hydrogenation of the adiponitrile can be carried out according to any process known in the art.

The following examples are given by way of non-limiting illustration of the present invention, and variations thereof that are readily accessible to a person skilled in the art.

### EXAMPLES

### Example 1

0.604 kg/h of dehydrated HMD with a content of water<500 ppm and an iUV of 0.13 was fed into a dividing wall column (Petlyuk column) with 65 theoretical plates. The top pressure of the column was controlled at 42 mbar. The bottom pressure was measured at 62 mbar. At the top of the column, the main part of lights compounds was extracted with a rate of 0.047 kg/h. 85% wt of HMD fed in the column was extracted in the side stream. The compounds with higher boiling points are removed from the bottom of the column.

iUV of the top stream was 0.09.

iUV of the pure HMD (side stream) was 0.05.

### Example 2

0.900 kg/h of dehydrated HMD with a content of water<500 ppm, an iUV of 0.45 and 65 ppm of ACHOL was fed into the same column as in example 1.

The top pressure of the column was controlled at 203 mbar. The bottom pressure was measured at 205 mbar. At the top of the column, the main part of light compounds was extracted with a rate of 0.05 kg/h. 87% wt of HMD fed in the column was extracted in the side stream. The compounds with higher boiling points were removed from the bottom of the column.

iUV of the top stream was 0.055.

iUV of the pure HMD (side stream) was 0.046.

The concentration of ACHOL in the pure HMD was <5 ppm.

The concentration of ACHOL in the bottom stream was >800 ppm.

### Comparative Example 3

2037 kg/h of dehydrated HMD with a content of water<500 ppm, 50 ppm of ACHOL and 1650 ppm of DCH, is fed as a vapor phase into a first distillation column, called column 1, with 80 theoretical plates. The top pressure of column 1 is 40 mbar. The bottom pressure of column 1 is 240 mbar. At the top of column 1, the main part of light compounds is extracted with a rate of 7 kg/h. The bottom rate is subjected to a second distillation column, called column 2, with 70 theoretical plates. The compounds with higher boiling points are removed from the bottom of column 2. 98.5% wt of HMD fed in column 1 is extracted as pure HMD in the top of column 2 with a composition of 4 ppm of DCH and 1 ppm of ACHOL. The top pressure of column 2 is 40 mbar. The bottom pressure of column 2 is 207 mbar.

The energy at the reboiler of column 1 is estimated to 1569 MJ/t HMD.

The energy at the reboiler of column 2 is estimated to 941 MJ/t HMD.

The total energy consumption (column 1+column2) is estimated to 2510 MJ/t HMD.

### Example 4

2037 kg/h of dehydrated HMD with a content of water<500 ppm, 50 ppm of ACHOL and 1,650 ppm of DCH, is fed as a vapor phase into a dividing wall column (Petlyuk column) with 116 theoretical plates. The top pressure of the column is 40 mbar. The bottom pressure is 273 mbar. At the top of the column, the main part of light compounds is extracted with a rate of 7 kg/h. The compounds with higher boiling points are removed from the bottom of the column. 98.5% wt of HMD fed in the column is extracted as pure HMD in the side stream with a composition of 4 ppm of DCH and 1 ppm of ACHOL.

The energy consumption at the reboiler is estimated to 1866 MJ/t HMD, about 25% less than the consumption with two successive columns as described in comparative example 3.

1. A process for purifying hexamethylenediamine, the process comprising:
distilling a mixture comprising hexamethylenediamine and impurities in a dividing wall column; and
recovering purified hexamethylenediamine from a side stream of the dividing wall column,
wherein the mixture, prior to the distilling, comprises impurities at an amount such that the mixture has a UV index of at least 0.10, and
wherein the purified hexamethylenediamine has a UV index of 0.08 or less.

2. The process of claim 1, wherein the mixture prior to distillation comprises less than 5,000 ppm of water.

3. The process of claim 1, wherein the purified hexamethylenediamine has a UV index equal to or below 0.05.

4. The process of claim 1, wherein the mixture prior to distillation comprises from 10 to 5,000 ppm of 1,2-aminocyclohexanol, based on a weight of the hexamethylenediamine in the mixture.

5. The process of claim 1, wherein the purified hexamethylenediamine comprises less than 8 ppm of 1,2-aminocyclohexanol.

6. The process of claim 1, wherein a top pressure of the dividing wall column is in a range of from 0.1 to 25 kPa.

7. The process of claim 1, wherein a bottom pressure of the dividing wall column is in a range of from 0.1 to 40 kPa.

8. The process of claim 1, wherein a total number of theoretical plates of the dividing wall column is at least 50.

9. The process of claim 1, wherein at least 75%, of the hexamethylenediamine fed into the dividing wall column is recovered as purified hexamethylenediamine.

10. A process for preparing hexamethylenediamine, the process comprising:
a) hydrogenating adiponitrile in the presence of a hydrogenation catalyst to obtain a mixture comprising hexamethylenediamine, impurities, and water;
b) optionally removing at least part of the water from the mixture obtained in the hydrogenating a),
c) optionally removing part of the impurities having a higher boiling point than hexamethylenediamine from the mixture obtained in the hydrogenating a) or the optional removing b); and
d) purifying the mixture obtained in the hydrogenating a) or the optional removing b) or c) by the process of claim 1.

11. The process of claim 10, wherein the removing b) is conducted and the mixture obtained in the removing b) comprises less than 5,000 ppm of water.

12. The process of claim 10, which comprises the removing b) and the removing c).

13. The process of claim 10, wherein at least part of a fraction obtained from the bottom of the dividing wall column in the purifying d) and/or at least part of the fraction comprising impurities having a higher boiling point than hexamethylenediamine obtained in the removing c) are treated to recover hexamethylenediamine, and the recovered hexamethylenediamine is recycled after the hydrogenating a).

14. The process of claim 1, wherein the mixture prior to distillation comprises less than 3,000 ppm of water.

15. The process of claim 1, wherein the mixture prior to distillation comprises less than 1,000 ppm of water.

16. The process of claim 1, wherein the mixture prior to distillation comprises less than 500 ppm of water.

17. The process of claim 1, wherein the mixture prior to distillation comprises from 10 to 400 ppm of 1,2-aminocyclohexanol, based on a weight of the hexamethylenediamine in the mixture.

18. The process of claim 1, wherein the purified hexamethylenediamine comprises less than 5 ppm of 1,2-aminocyclohexanol.

19. The process of claim 1, wherein the purified hexamethylenediamine comprises less than 2 ppm of 1,2-aminocyclohexanol.
